(19) United States
(12) Patent Application Publication
(10) Pub. No.: US 2026/0074309 A1
(43) Pub. Date: Mar. 12, 2026

(54) HYDROGEN SULFIDE DETECTION DEVICE

(71) Applicant: TOYOTA JIDOSHA KABUSHIKI KAISHA, Toyota-shi (JP)

(72) Inventors: Masaya MURASE, Nagoya-shi (JP); Yuji NISHI, Nagoya-shi (JP)

(73) Assignee: TOYOTA JIDOSHA KABUSHIKI KAISHA, Toyota-shi (JP)

(21) Appl. No.: 19/234,702

(22) Filed: Jun. 11, 2025

(30) Foreign Application Priority Data
Sep. 11, 2024 (JP) 2024-157341

(51) Int. Cl. H01M 10/42; G01N 27/02; G01N 33/00; H01M 10/48

(52) U.S. Cl. CPC H01M 10/4285; G01N 27/021; G01N 33/0044; H01M 10/48

(57) ABSTRACT

A hydrogen sulfide detection device includes a wire that electrically connects a first node and a second node, and a monitoring circuit that monitors a voltage between the first node and the second node. The wire includes a plurality of metal exposed portions in which a metal that reacts with the hydrogen sulfide to corrode is exposed.

### CROSS-REFERENCE TO RELATED APPLICATION

This application claims priority to Japanese Patent Application No. 2024-157341 filed on Sep. 11, 2024. The disclosure of the above-identified application, including the specification, drawings, and claims, is incorporated by reference herein in its entirety.

### BACKGROUND

### 1. Technical Field

The present disclosure relates to a hydrogen sulfide detection device to be applied to a battery pack that stores a battery cell including a sulfide-based electrolyte.

### 2. Description of Related Art

An all solid state battery is attracting attention as a next-generation battery cell configuring a battery pack. The all solid state battery has advantages of high safety and long lifetime as compared with conventional batteries using liquid as an electrolyte. In particular, an all solid state battery using a sulfide-based electrolyte has a large capacity and high output, and is expected to be used as a battery for a vehicle.

Meanwhile, when the battery cell including the sulfide-based electrolyte is configured as the battery cell of the all solid state battery, a failure may cause generation of a hydrogen sulfide gas. The hydrogen sulfide gas is toxic, and corrodes surrounding metal components. Accordingly, there is a demand for a technology for appropriately detecting generation of hydrogen sulfide in the battery pack that stores the battery cell including the sulfide-based electrolyte.

Japanese Unexamined Patent Application Publication No. 2017-199667 (JP 2017-199667 A) discloses a detection system in which a resistance change member containing a resistance change material of which electrical resistance is changed by a chemical reaction with hydrogen sulfide is provided in a battery cell, and whether or not hydrogen sulfide is generated in the battery cell is determined based on a detection value between terminals of the resistance change member. Additionally, the following WO 2003/029801 is provided as a document representing the technical level of the technical field.

### SUMMARY

In the battery cell including the sulfide-based electrolyte serving as the all solid state battery, it is extremely difficult to predict a portion of generation of hydrogen sulfide in advance. Further, it is also difficult to predict how the generated hydrogen sulfide is to be distributed. Accordingly, in order to appropriately detect the generation of hydrogen sulfide, it is desired to perform detection at a plurality of portions in the battery pack.

However, in the technology disclosed in JP 2017-199667 A, a detection circuit is configured for each resistance change member, and hence the detection circuit becomes complicated when resistance change members are disposed at a plurality of portions in the battery pack. Accordingly, when detection is performed at a plurality of portions in the battery pack, the increase in cost becomes a problem. Further, a space required for configuring the detection system is also increased.

The present disclosure has been made in view of the above-mentioned problems. The present disclosure has one object to provide a technology capable of detecting generation of hydrogen sulfide at a plurality of portions in a battery pack while reducing cost.

One aspect of the present disclosure relates to a hydrogen sulfide detection device to be applied to a battery pack that stores a battery cell including a sulfide-based electrolyte. The hydrogen sulfide detection device includes a wire that electrically connects a first node and a second node, and a monitoring circuit that monitors a voltage between the first node and the second node. The wire includes a plurality of metal exposed portions in which a metal that reacts with hydrogen sulfide to corrode is exposed.

With the present disclosure, the metal exposed portions are disposed at a plurality of portions in the battery pack, and thus the generation of hydrogen sulfide can be detected at a plurality of portions of the battery pack. Further, with the present disclosure, regardless of the number of detection portions, the hydrogen sulfide detection device is configured of the monitoring circuit and one wire, and hence the cost can be reduced.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic diagram illustrating a configuration of a hydrogen sulfide detection device according to a first embodiment;

FIG. 2A is an explanatory schematic diagram illustrating an operation of the hydrogen sulfide detection device;

FIG. 2B is an explanatory schematic diagram illustrating the operation of the hydrogen sulfide detection device;

FIG. 3 is a flowchart illustrating a processing flow of processing executed by a monitoring circuit;

FIG. 4 is a conceptual diagram illustrating an example of arrangement of metal exposed portions;

FIG. 5 is a schematic diagram illustrating a configuration of a hydrogen sulfide detection device according to a second embodiment; and

FIG. 6 is a schematic diagram illustrating a configuration of a hydrogen sulfide detection device according to a third embodiment.

### DETAILED DESCRIPTION OF EMBODIMENTS

Hereinafter, embodiments of the present disclosure are described with reference to the accompanying drawings. It is to be noted that the same or corresponding configurations in each figure are denoted by the same reference symbols, and description thereof is simplified or omitted.

### 1 First Embodiment

### 1.1 Configuration

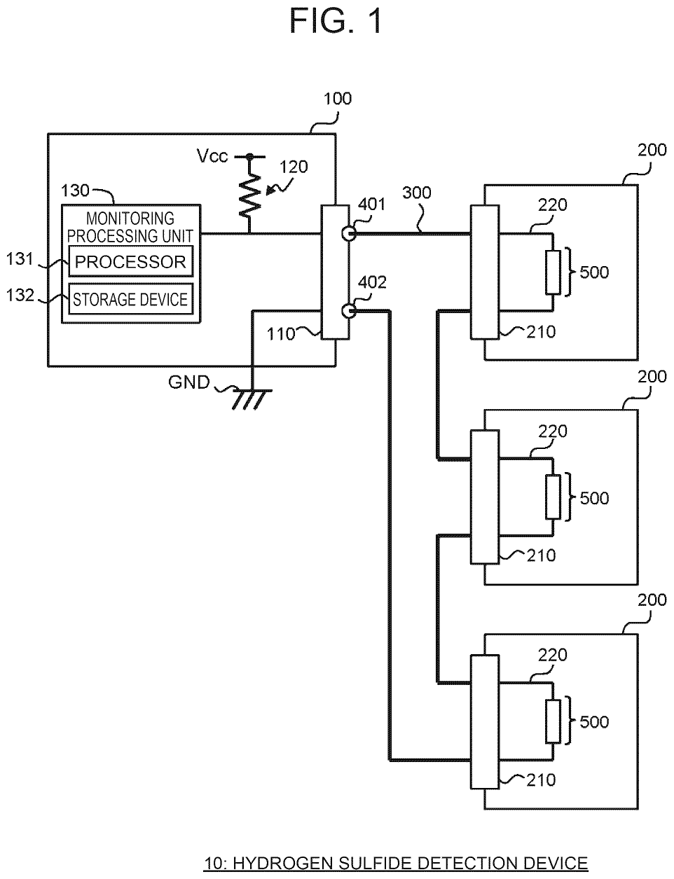

FIG. 1 is a schematic diagram illustrating a configuration of a hydrogen sulfide detection device 10 according to a first embodiment. The hydrogen sulfide detection device 10 is applied to a battery pack that stores a battery cell including a sulfide-based electrolyte, and detects generation of hydrogen sulfide. The hydrogen sulfide detection device 10 is stored in the battery pack together with the battery cell. The battery cell including the sulfide-based electrolyte is typically an all solid state battery using a solid sulfide-based electrolyte. The mode of the battery cell is not particularly limited. For example, the mode of the battery cell may be a laminated type or a prismatic type. The all solid state battery using the sulfide-based electrolyte has a large capacity and high output, and is suitable for a battery of a vehicle. Thus, the battery pack to which the hydrogen sulfide detection device 10 is applied may particularly be a battery to be mounted on a vehicle.

The hydrogen sulfide detection device 10 includes a monitoring circuit 100 and a plurality of substrates 200. The substrates 200 are printed circuit boards (PCB) having the same pattern 220 formed of a metal. Each of the substrates 200 may particularly be a flexible printed circuit (FPC).

The monitoring circuit 100 is connected to an external device via a connector 110, and the substrates 200 are each connected to an external device via a connector 210. As illustrated in FIG. 1, the monitoring circuit 100 and the substrates 200 are connected in daisy chain connection by a cable 300. In this manner, the cable 300 and the pattern 220 of each of the substrates 200 form a wire that electrically connects a first node 401 and a second node 402 (hereinafter simply referred to as “wire”). The wire forms one current path.

The monitoring circuit 100 monitors a voltage between the first node 401 and the second node 402. In the monitoring circuit 100, the first node 401 is connected to a power supply of a voltage Vcc (for example, 5 V) via a resistor 120, and the second node 402 is connected to a ground GND having a reference potential (for example, 0 V). The monitoring circuit 100 includes a monitoring processing unit 130. The monitoring processing unit 130 is a computer that executes processing of monitoring the voltage. The monitoring processing unit 130 may particularly be a microcontroller. The monitoring processing unit 130 is disposed to receive a potential between the resistor 120 and the first node 401 as input. For example, when the monitoring processing unit 130 is a microcontroller, an input port of the microcontroller is connected between the resistor 120 and the first node 401. The resistor 120 is a pull-up resistor for the monitoring processing unit 130. For example, the resistance value of the resistor 120 is about 10 kΩ. The second node 402 is connected to the ground GND, and hence the monitoring processing unit 130 can detect the voltage between the first node 401 and the second node 402.

It is to be noted that the voltage between the first node 401 and the second node 402 can be indirectly detected even by measuring a voltage across both ends of the resistor 120. Thus, monitoring the voltage between the first node 401 and the second node 402 includes measuring the voltage across both ends of the resistor 120. From this viewpoint, the monitoring processing unit 130 may be disposed to measure the voltage across both ends of the resistor 120.

The monitoring processing unit 130 includes one or more processors 131 (hereinafter simply referred to as “processor 131”) and one or more storage devices 132 (hereinafter simply referred to as “storage device 132”). The processor 131 executes various types of processing. Examples of the processor 131 include a general-purpose processor, a specific-use processor, a central processing unit (CPU), a graphics processing unit (GPU), an application specific integrated circuit (ASIC), a field-programmable gate array (FPGA), an integrated circuit, a conventional circuit, and one or more combinations thereof. The processor 131 can also be called a processing circuitry. The storage device 132 stores various types of information required for execution of processing by the processor 131. Examples of the storage device 132 include recording media such as a random access memory (RAM), a read only memory (ROM), a solid state drive (SSD), and a hard disk drive (HDD). The storage device 132 stores a computer program that can be executed by the processor 131. The computer program is configured of a plurality of instruction codes writing the processing to be executed by the processor 131. The computer program is recorded in a computer-readable recording medium. The functions of the monitoring processing unit 130 are implemented through cooperation between the storage device 132 and the processor 131 that executes the computer program.

As a metal forming the pattern 220 of the substrate 200, a metal that reacts with hydrogen sulfide to corrode is used. For example, copper or silver is used as the metal. Further, in the first embodiment, a part of the pattern 220 of each of the substrates 200 forms a metal exposed portion 500 by exposing the metal to the surface of the substrate 200. This state can be achieved by configuring the substrate 200 so as to prevent surface protection (for example, solder resist or coverlay) or surface treatment (for example, plating) from being performed in the part of the pattern 220. As described above, in the first embodiment, a part of the pattern 220 of each of the substrates 200 forms the metal exposed portion 500. In this manner, as illustrated in FIG. 1, the wire includes a plurality of metal exposed portions 500.

### 1.2 Operation of Hydrogen Sulfide Detection Device

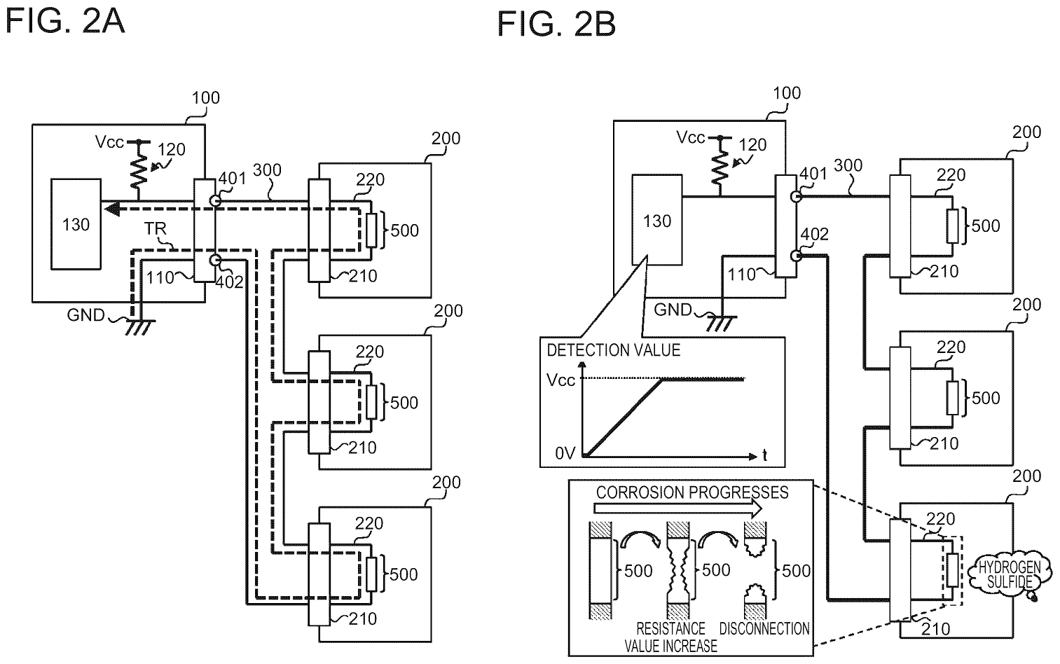

Hereinafter, an operation of the hydrogen sulfide detection device 10 according to the first embodiment is described. FIG. 2A and FIG. 2B are explanatory schematic diagrams illustrating the operation of the hydrogen sulfide detection device 10.

FIG. 2A illustrates an operation when the target battery pack is normal. That is, FIG. 2A illustrates an operation when no hydrogen sulfide is generated from the battery cell. When the battery pack is normal, the wire is simply a current path including no resistor. Thus, as shown in a transmission path TR, the potential of the ground GND is directly input to the monitoring processing unit 130. That is, the monitoring processing unit 130 acquires the reference potential as a detection value.

FIG. 2B illustrates an operation when hydrogen sulfide is generated from the battery cell. At this time, the metal of the metal exposed portion 500 reacts with the generated hydrogen sulfide to corrode. When the metal becomes a sulfide by corrosion, the resistance value of the metal exposed portion 500 increases. Moreover, the metal tends to move radially by corrosion. Further, the corroded metal moves also when vibrations are applied thereto. For example, when vibrations of the vehicle are transmitted, the corroded metal moves. As the corrosion progresses as described above, the metal of the metal exposed portion 500 gradually disappears to reduce its sectional area. In this manner, the resistance value of the metal exposed portion 500 further increases. Then, the metal of the metal exposed portion 500 is finally disconnected.

As described above, when hydrogen sulfide is generated from the battery cell, the resistance value of the metal exposed portion 500 gradually increases, and the metal exposed portion 500 is finally disconnected. Thus, in a process in which the resistance value of the metal exposed portion 500 increases, the monitoring processing unit 130 acquires a divided voltage caused by the resistance value of the metal exposed portion 500 as the detection value. That is, as the resistance value of the metal exposed portion 500 increases, the detection value of the monitoring processing unit 130 increases from the reference potential. Then, when the metal exposed portion 500 is finally disconnected, the voltage Vcc of the power supply is directly input to the monitoring processing unit 130. That is, the monitoring processing unit 130 acquires the voltage Vcc of the power supply as the detection value.

FIG. 2B illustrates, in a graph, an example of the detection value of the monitoring processing unit 130 when the reference potential is 0 V. As illustrated in the graph, when the hydrogen sulfide is generated from the battery cell, the detection value of the monitoring processing unit 130 changes from 0 V to Vcc. Thus, the monitoring processing unit 130 can determine whether or not the hydrogen sulfide is generated from the battery cell based on the detection value (the voltage between the first node 401 and the second node 402). For example, the monitoring processing unit 130 determines that the hydrogen sulfide is generated from the battery cell in response to the fact that a variation amount from an initial value of the detection value has become larger than a threshold value. Further, for example, the monitoring processing unit 130 may determine that the hydrogen sulfide is generated from the battery cell without calculating the variation amount of the detection value but directly using the detection value, in response to the fact that the detection value has become larger than a threshold value.

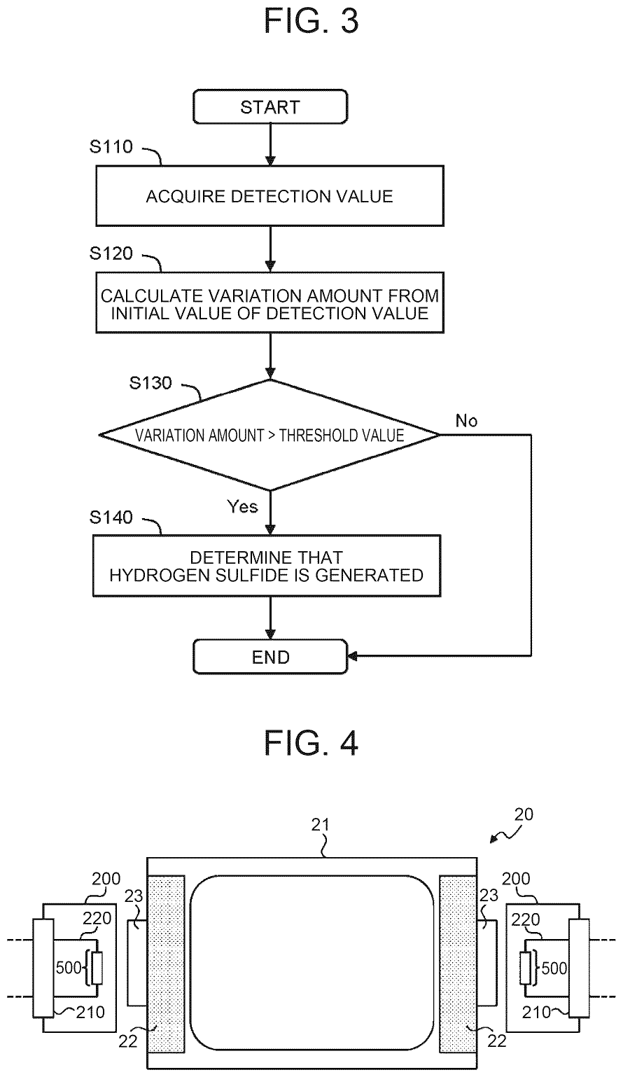

FIG. 3 is a flowchart illustrating a processing flow of processing executed by the monitoring circuit 100 (in more detail, the monitoring processing unit 130). The processing flow illustrated in FIG. 3 is repeatedly executed for each predetermined processing period.

First, in step S110, the monitoring circuit 100 acquires the detection value. Next, in step S120, the monitoring circuit 100 calculates the variation amount from the initial value of the detection value. In the hydrogen sulfide detection device 10, the initial value of the detection value is the reference potential, and the variation amount from the initial value is a difference between the detection value and the reference potential. In particular, when the reference potential is 0 V, the variation amount from the initial value coincides with the detection value. Next, in step S130, the monitoring circuit 100 determines whether or not the calculated variation amount is larger than a threshold value.

When the variation amount is equal to or lower than the threshold value (step S130; No), the monitoring circuit 100 determines that no hydrogen sulfide is generated, and ends the processing of this time. When the variation amount is larger than the threshold value (step S130; Yes), the monitoring circuit 100 determines that hydrogen sulfide is generated from the battery cell (step S140). The monitoring circuit 100 may further execute processing of warning the user about the generation of the hydrogen sulfide by display or sound.

### 1.3 Effects

As described above, the hydrogen sulfide detection device 10 according to the first embodiment can detect generation of hydrogen sulfide from the battery cell. In particular, with the hydrogen sulfide detection device 10, the generation of the hydrogen sulfide is detected when the metal of any one of the metal exposed portions 500 reacts with the hydrogen sulfide. That is, the hydrogen sulfide detection device 10 can detect the generation of the hydrogen sulfide at a plurality of portions in the battery pack by disposing the metal exposed portions 500 at a plurality of portions in the battery pack. In addition, the hydrogen sulfide detection device 10 is configured of one monitoring circuit 100 and one wire serving as a current path regardless of the number of detection portions. In particular, the hydrogen sulfide detection device 10 can be achieved by using only one input port of the monitoring processing unit 130 of the monitoring circuit 100. As described above, the hydrogen sulfide detection device 10 can be configured with low cost and saved space without causing the circuit to become complicated even when the number of detection portions is increased. Moreover, as compared with the case where the detection value between the terminals of the battery cell is used, when the circuit is not electrically connected to the battery cell, the circuit configuration can be simplified, and the expansion of the detection portions can be easily performed.

Further, according to the first embodiment, the metal exposed portion 500 is formed by the pattern 220 of the substrate 200. The pattern 220 of the substrate 200 can be formed to be very thin. For example, the pattern 220 can be formed with a thickness of about 50 μm. When the metal exposed portion 500 is formed with such a pattern 220, the progression speed of corrosion when the metal of the metal exposed portion 500 reacts with the hydrogen sulfide can be increased. That is, when the hydrogen sulfide is generated from the battery cell, the metal of the metal exposed portion 500 quickly disappears and is easily disconnected. As a result, the detection accuracy of the hydrogen sulfide detection device 10 can be enhanced. Further, the hydrogen sulfide detection device 10 can be configured by re-using an existing substrate used for voltage monitoring of the battery cell or the like. In this manner, the cost can be further reduced.

Moreover, according to the first embodiment, the hydrogen sulfide detection device 10 includes the substrates 200 corresponding to the respective metal exposed portions 500. Each of the metal exposed portions 500 is formed by the pattern 220 of the corresponding substrate 200 out of the substrates 200. In other words, the metal exposed portions 500 are formed by the respective patterns 220 of the separate substrates 200. In addition, the substrates 200 are connected in daisy chain connection by the cable 300. In this manner, the metal exposed portions 500 can be individually disposed at desired locations within the battery pack. As a result, the hydrogen sulfide detection device 10 having a high degree of freedom in disposing the detection portion can be achieved.

Incidentally, it is highly possible that the portion in the battery cell at which the hydrogen sulfide is to be generated is a sealing portion (seal portion) of an exterior member of the battery cell. The reason therefor is because the sealing portion is likely to be reduced in durability against scratches than other parts. In view of the above, the metal exposed portions 500 may include a metal exposed portion 500 disposed adjacent to the sealing portion of the exterior member of the battery cell.

FIG. 4 is a conceptual diagram illustrating an example of arrangement of the metal exposed portions 500. FIG. 4 schematically illustrates a battery cell 20. When the battery cell 20 is of a laminated type, an exterior member 21 of the battery cell 20 is typically a laminate film. Further, when the battery cell 20 is of a prismatic type, the exterior member 21 of the battery cell 20 is typically a metal can. In the example illustrated in FIG. 4, a sealing portion 22 of the exterior member 21 is a position covering an electrode terminal 23 of the battery cell 20. In addition, the substrate 200 is disposed adjacent to the sealing portion 22 of the exterior member 21. As described above, the metal exposed portion 500 is disposed adjacent to the sealing portion 22 of the exterior member 21 of the battery cell 20.

As described above, when the metal exposed portion 500 is disposed adjacent to the sealing portion 22 of the exterior member 21 of the battery cell 20, the detection accuracy of the hydrogen sulfide detection device 10 can be enhanced.

### 1.4 Modification Example

In the above-mentioned first embodiment, the monitoring circuit 100 is configured so that the resistor 120 becomes a pull-up resistor for the monitoring processing unit 130. As a modification example, the monitoring circuit 100 may be configured so that the resistor 120 becomes a pull-down resistor for the monitoring processing unit 130. That is, the first node 401 may be directly connected to the power supply, and the second node 402 may be connected to the ground GND via the resistor 120. Further, the monitoring processing unit 130 may be disposed to detect the voltage between the first node 401 and the second node 402. For example, the input port of the monitoring processing unit 130 is connected between the resistor 120 and the second node 402.

With the hydrogen sulfide detection device 10 according to the modification example, at the normal time, the voltage Vcc of the power supply is directly input to the monitoring processing unit 130. That is, the monitoring processing unit 130 acquires the voltage Vcc as the detection value. Then, when hydrogen sulfide is generated from the battery cell, similarly to the above-mentioned operation, the resistance value of the metal exposed portion 500 increases, and the metal exposed portion 500 is finally disconnected. In this manner, the detection value of the monitoring processing unit 130 changes from Vcc to 0 V. Thus, also in the modification example, the monitoring processing unit 130 can determine whether or not hydrogen sulfide is generated from the battery cell based on the detection value. The processing flow of the processing executed by the monitoring circuit 100 at this time may be the same as that illustrated in FIG. 3. In the hydrogen sulfide detection device 10 according to the modification example, the initial value of the detection value is the voltage Vcc of the power supply, and the variation amount from the initial value is a difference between the detection value and Vcc.

As described above, even with the hydrogen sulfide detection device 10 according to the modification example, effects similar to the above-mentioned effects can be provided.

### 2 Second Embodiment

Hereinafter, a second embodiment is described. It is to be noted that, in the following, the difference from the first embodiment is mainly described, and description of content overlapping the first embodiment is omitted as appropriate.

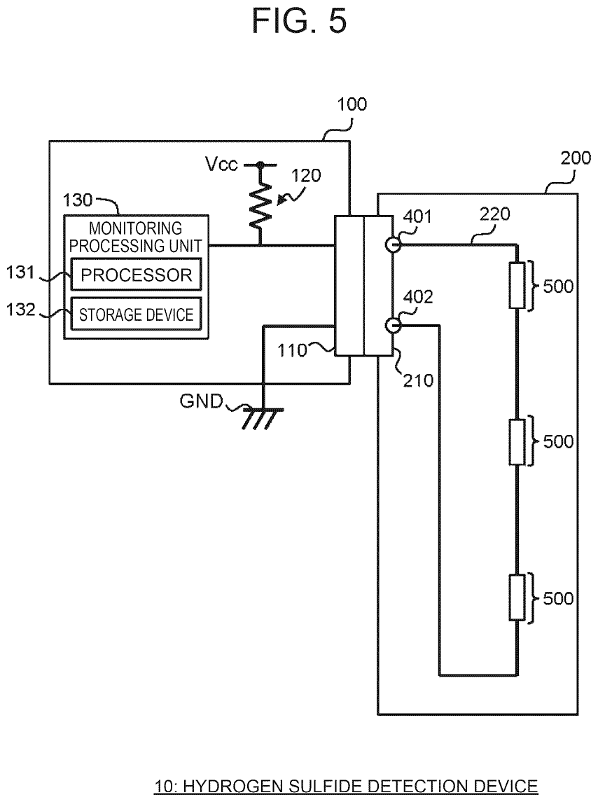

FIG. 5 is a schematic diagram illustrating the configuration of the hydrogen sulfide detection device 10 according to the second embodiment. The hydrogen sulfide detection device 10 according to the second embodiment includes the monitoring circuit 100 similarly to the first embodiment. Meanwhile, as compared with the first embodiment, in the second embodiment, the hydrogen sulfide detection device 10 includes only one substrate 200. The monitoring circuit 100 and the substrate 200 are directly connected by the connectors 110, 210. In the second embodiment, the pattern 220 of the substrate 200 forms the wire that electrically connects the first node 401 and the second node 402.

In the second embodiment, the pattern 220 of the substrate 200 forms the metal exposed portions 500 in which metal is exposed from the surface of the substrate 200 at a plurality of portions. In this manner, as illustrated in FIG. 5, the wire includes the metal exposed portions 500.

The operation of the hydrogen sulfide detection device 10 according to the second embodiment is the same as the first embodiment. Thus, the hydrogen sulfide detection device 10 according to the second embodiment can detect the generation of the hydrogen sulfide at a plurality of portions in the battery pack by disposing the metal exposed portions 500 at a plurality of portions in the battery pack, similarly to the first embodiment. Further, the hydrogen sulfide detection device 10 according to the second embodiment can be configured with low cost and saved space without causing the circuit to become complicated even when the number of detection portions is increased, similarly to the first embodiment.

In the hydrogen sulfide detection device 10 according to the second embodiment, the metal exposed portions 500 are formed by the pattern 220 of the same substrate 200, and hence the degree of freedom in disposing the detection portion is reduced than that in the first embodiment. Meanwhile, the hydrogen sulfide detection device 10 according to the second embodiment can be configured of one substrate 200, and no cable 300 for connection is required. Thus, the cost can be reduced as compared with the first embodiment.

### 3 Third Embodiment

Hereinafter, a third embodiment is described. It is to be noted that, in the following, the difference from the first embodiment is mainly described, and description of content overlapping the first embodiment is omitted as appropriate.

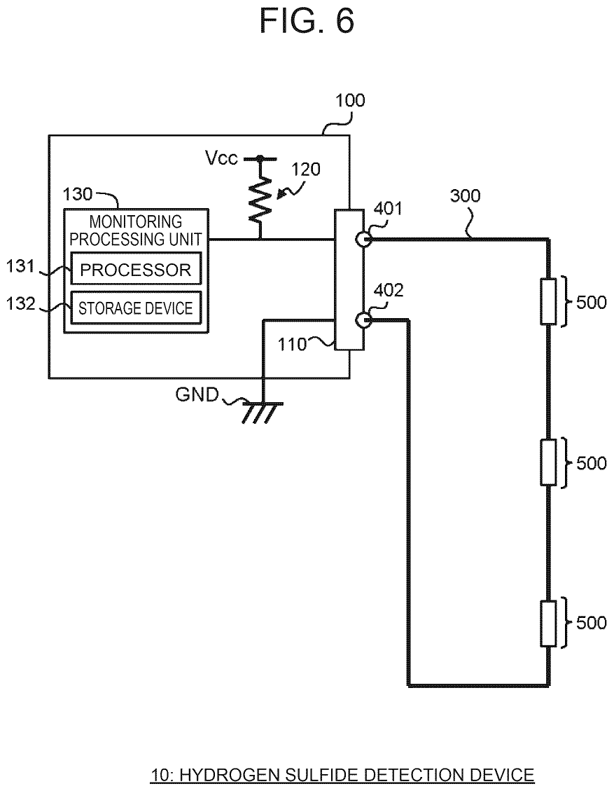

FIG. 6 is a schematic diagram illustrating the configuration of the hydrogen sulfide detection device 10 according to the third embodiment. The hydrogen sulfide detection device 10 according to the third embodiment includes the monitoring circuit 100 similarly to the first embodiment. Meanwhile, as compared with the first embodiment, in the third embodiment, the hydrogen sulfide detection device 10 includes no substrate 200. In place of the substrate 200, both ends of the one cable 300 are connected to the connector 110 of the monitoring circuit 100. In the third embodiment, the cable 300 forms the wire that electrically connects the first node 401 and the second node 402.

In the third embodiment, as the metal configuring the cable 300, a metal that reacts with hydrogen sulfide to corrode is used. Further, the cable 300 includes metal exposed portions 500 in which the metal is exposed to the outside of the cable 300 at a plurality of portions. This state can be achieved by peeling off the covering of the cable 300 at a plurality of portions. In this manner, as illustrated in FIG. 6, the wire includes the metal exposed portions 500.

The operation of the hydrogen sulfide detection device 10 according to the third embodiment is the same as the first embodiment. Thus, the hydrogen sulfide detection device 10 according to the third embodiment can detect the generation of the hydrogen sulfide at a plurality of portions in the battery pack by disposing the metal exposed portions 500 at a plurality of portions in the battery pack, similarly to the first embodiment. Further, the hydrogen sulfide detection device 10 according to the third embodiment can be configured with low cost and saved space without causing the circuit to become complicated even when the number of detection portions is increased, similarly to the first embodiment.

In the hydrogen sulfide detection device 10 according to the third embodiment, the metal exposed portion 500 cannot be formed very thin unlike the pattern 220 of the substrate 200, and hence the detection accuracy is reduced as compared with the first embodiment. Meanwhile, the hydrogen sulfide detection device 10 according to the third embodiment can be easily configured of one cable 300 without requiring designing of the substrate 200, as compared with the first embodiment. Further, the cost can be reduced.

What is claimed is:

1. A hydrogen sulfide detection device to be applied to a battery pack that stores a battery cell including a sulfide-based electrolyte, the hydrogen sulfide detection device comprising:
a wire that electrically connects a first node and a second node; and
a monitoring circuit that monitors a voltage between the first node and the second node, wherein:
the wire includes a plurality of metal exposed portions in which a metal that reacts with hydrogen sulfide to corrode is exposed.

2. The hydrogen sulfide detection device according to claim 1, wherein the metal exposed portions include a pattern of at least one substrate.

3. The hydrogen sulfide detection device according to claim 2, wherein:
the at least one substrate includes a plurality of substrates corresponding to the respective metal exposed portions; and
the metal exposed portions each include the pattern of a corresponding substrate out of the substrates.

4. The hydrogen sulfide detection device according to claim 1, wherein the metal exposed portions include a metal exposed portion disposed adjacent to a sealing portion of an exterior member of the battery cell.

5. The hydrogen sulfide detection device according to claim 1, wherein the monitoring circuit determines whether or not the hydrogen sulfide is generated from the battery cell based on a change in the voltage.
